(19) United States
(12) Patent Application Publication
(10) Pub. No.: US 2026/0294550 A1
(43) Pub. Date: Oct. 1, 2026

(54) METHOD AND APPARATUS FOR PROCESSING SURGICAL INFORMATION BASED ON ARTIFICIAL INTELLIGENCE

(71) Applicant: KOREA INSTITUTE OF SCIENCE AND TECHNOLOGY, Seoul (KR)

(72) Inventors: Juyoun PARK, Seoul (KR); Taemin CHOI, Seoul (KR); Yong Seok IHN, Seoul (KR)

(73) Assignee: KOREA INSTITUTE OF SCIENCE AND TECHNOLOGY, Seoul (KR)

(21) Appl. No.: 19/569,588

(22) Filed: Mar. 17, 2026

(30) Foreign Application Priority Data
Mar. 28, 2025 (KR) 10-2025-0040385

(51) Int. Cl. A61B 34/20; A61B 90/00; G06F 40/279; G06T 7/00; G06T 7/73; G06V 10/26; G06V 10/44; G06V 10/62; G06V 10/764; G06V 20/40; G06V 20/50; G16H 50/70

(52) U.S. Cl. CPC A61B 34/20; G06F 40/279; G06T 7/0012; G06T 7/73; G06V 10/26; G06V 10/44; G06V 10/62; G06V 10/764; G06V 20/41; G06V 20/46; G06V 20/50; G16H 50/70; A61B 2034/2055; A61B 2034/2065; A61B 2090/0805; G06T 2207/30004; G06V 2201/034

(57) ABSTRACT

The present disclosure relates to an image processing technology, and a method of processing surgical information includes receiving a surgical image and surgical-related text, extracting an image feature vector from the received surgical image and extracting at least two text feature vectors from the received text, generating a composite feature vector from the image feature vector and the text feature vector, and detecting an instrument present in the surgical image using the generated composite feature vector.

### CROSS-REFERENCE TO RELATED APPLICATIONS

This application claims the benefit of Korea Patent Application No. 10-2025-0040385, filed on Mar. 28, 2025, which is incorporated herein by reference for all purposes as if fully set forth herein.

### TECHNICAL FIELD

The present disclosure relates to an image processing technology, and more specifically, to a method of processing surgical information based on artificial intelligence, a recording medium recording the method, and an apparatus for processing surgical information, in which surgical instruments are detected from surgical images or videos, a surgical procedure is classified or predicted using the detection results, and supplementary information related to the surgery is provided to medical staff.

### BACKGROUND

In modern industry, computer vision technology using artificial intelligence (AI) is actively used in various fields. In particular, technologies such as object detection, image classification, and image segmentation are becoming essential components in various industries, including manufacturing, autonomous driving, security, healthcare, etc. The advancement of these technologies is accelerating with the advent of AI-based deep neural networks (DNNs) and the availability of large-scale datasets, enabling more sophisticated and accurate image processing results. Segmentation is a crucial computer vision technology that segments objects within an image in units of pixel and enables the effective isolation and analysis of specific objects or regions within a given image. In particular, segmentation technology is being actively used in the field of medical image analysis.

Meanwhile, minimally invasive surgeries (MIS) are widely used due to their numerous advantages, allowing surgeons to perform a surgical procedure based on images captured by a camera rather than relying on direct visual observation. In these surgical environments, surgical instrument segmentation (SIS) is an essential technology for enhancing visual recognition. The patent document to be presented below introduces a technical configuration for surgical instrument segmentation. Deep learning-based surgical instrument segmentation methods have demonstrated a certain level

of performance, but still have performance limitations due to small datasets and lack adaptability to human-robot interaction in robot-assisted surgery. To solve such a problem, research on multi-modal data processing methods that use both image and text inputs has been actively pursued. Conventional segmentation models primarily use only image data, but in the case of medical image analysis, text data related to relevant medical information may also be provided. Effectively processing this multi-modal data is expected to improve the accuracy of segmentation models. Accordingly, a new segmentation approach combining image and text information is required, and the design and optimization of AI models for this purpose are emerging as important research challenges.

### DOCUMENTS OF RELATED ART

[Non-patent documents] A. A. Shvets, A. Rakhlin, A. A. Kalinin, and V. I. Iglovikov, “Automatic instrument segmentation in robot-assisted surgery using deep learning,” in 2018 17th IEEE international conference on machine learning and applications (ICMLA). IEEE, 2018, pp. 624-628

### SUMMARY

Various embodiments of the present disclosure are directed to overcoming performance limitations of a conventional single-image-based surgical instrument segmentation model, to solving the problem of generating an incorrect mask even when an instrument is not actually present in a multi-modal-based surgical instrument segmentation model that uses both image input and text input, and to minimize the risk of accidents that may occur during a surgical procedure performed by medical staff based on images in an environment with a limited field of view, such as minimally invasive surgery. According to one embodiment of the present disclosure, there is provided a method of processing,

by an apparatus for processing surgical information including at least one processor, the surgical information, which includes receiving a surgical image and surgical-related text, extracting an image feature vector from the received surgical image and extracting at least two text feature vectors from the received text, generating a composite feature vector from the image feature vector and the text feature vector, and detecting an instrument present in the surgical image using the generated composite feature vector.

The generating of the composite feature vector may include generating a first composite feature vector from the image feature vector and a first text feature vector related to an instrument to generate a first mask for the instrument determined to be present in the surgical image, and generating a second composite feature vector from the image feature vector and a second text feature vector related to a location of the instrument determined to be present in the surgical image to generate a second mask for the instrument determined to be present in the surgical image.

The generating of the first mask may include generating a first composite feature vector by performing multi-modal fusion of a first text feature vector extracted based on at least one text input related to a name or attribute of an instrument and the image feature vector, and generating a segmentation mask corresponding to an instrument whose probability of being present in the surgical image is greater than or equal to a first reference value, among instruments input via text by referencing the generated first composite feature vector.

The generating of the second mask may include generating a second composite feature vector by performing multi-modal fusion of the second text feature vector extracted based on text input regarding the location of the instrument derived using the first mask and the image feature vector, and generating a segmentation mask corresponding to an instrument whose probability of being present in the surgical image is greater than or equal to a second reference value, among instruments input via text by referencing the generated second composite feature vector.

The detecting of the instrument present in the surgical image may include calculating a single mask by fusing a plurality of masks generated for an instrument determined to be present in the surgical image using the generated composite feature vector, and outputting a segmentation map for the instrument present in the surgical image based on the calculated single mask.

The method may further include acquiring time-series feature information by extracting feature vectors from individual surgical images constituting a surgical video, and classifying a current surgical procedure from the acquired time-series feature information.

The surgical procedure may follow a hierarchical structure of a stage, a phase, and a step according to a classification granularity and is classified using a single hierarchy or a combination of two or more, and the classifying of the current surgical procedure may include using at least one hierarchical classifier based on a fusion model of spatial visual information and temporal flow information within the image to output a surgical procedure with a highest probability value from the time-series feature information as a classification result.

The method may further include receiving the acquired time-series feature information and the classified current surgical procedure and predicting a next surgical procedure using at least one of locations, distances, and relationships among instruments detected in a current surgical image.

The predicting of the next surgical procedure may include referencing the fusion model to determine a surgical procedure having a highest probability of appearing temporally consecutively to the current surgical procedure as the next surgical procedure and outputting at least one of the determined next surgical procedure or a remaining time until the next surgical procedure.

The method may further include deriving feature information for each of a plurality of surgical procedures constituting a surgery and storing the feature information together with temporal information in advance as a standard operating procedure for a surgical procedure, and comparing the time-series feature information acquired from the surgical video and the classified current surgical procedure with the pre-stored standard operating procedure to evaluate at least one of precision, skill, completion, efficiency, and safety of a surgery.

Hereinafter, there is provided a computer-readable recording medium storing a program for executing the above-described method on a computer.

According to one embodiment of the present disclosure, there is provided an apparatus for processing task information, which includes a memory configured to store a program for processing task information, and a processor configured to execute the program stored in the memory, wherein the program includes instructions to receive a task image and task-related text, extract an image feature vector from the received task image and extract at least two text feature vectors from the received text, generate a composite feature vector from the image feature vector and the text feature vector, and detect an instrument present in the task image using the generated composite feature vector.

The program may perform instructions to generate the composite feature vector by generating a first composite feature vector from the image feature vector and a first text feature vector related to an instrument to generate a first mask for an instrument determined to be present in the task image, and generating a second composite feature vector from the image feature vector and the second text feature vector relating to a location of the instrument determined to be present in the task image to generate a second mask for the instrument determined to be present in the task image.

The program may perform instructions to generate the first mask by generating the first composite feature vector by performing multi-modal fusion of a first text feature vector extracted based on at least one text input related to a name or attribute of an instrument and the image feature vector, and generating a segmentation mask corresponding to an instrument whose probability of presence in the task image is greater than or equal to a first reference value among instruments input via text by referencing the generated first composite feature vector.

The program may perform instructions to generate the second mask by generating a second composite feature vector by performing multi-modal fusion of the second text feature vector extracted based on text input regarding the location of the instrument derived using the first mask and the image feature vector, and generating a segmentation mask corresponding to an instrument whose probability of presence in the task image is greater than or equal to a second reference value among instruments input via text by referencing the generated second composite feature vector.

The program may perform instructions to detect the instrument present in the task image by calculating a single mask by fusing a plurality of masks generated for an instrument determined to be present in the task image using the generated composite feature vector, and outputting a segmentation map for the instrument present in the task based on the calculated single mask.

The program may further include instructions to acquire time-series feature information by extracting feature vectors from individual task images constituting a task video, and classify a current task process from the acquired time-series feature information.

The program may further include instructions to receive the acquired time-series feature information and the classified current task process and predict a next task procedure using at least one of locations, distances, and relationships among instruments detected in a current task image.

According to various embodiments of the present disclosure, in implementing the multi-modal based surgical instrument segmentation model that uses both image input and text input, the segmentation map can be generated through prompt-based iterative refinement without prior knowledge of the existence of objects in the image, thereby enabling detection of instruments that did not appear during learning, effectively preventing false positive problems, and enabling precise instrument detection even under robust conditions by balancing the vision-based model and the prompt-based model.

### BRIEF DESCRIPTION OF THE DRAWINGS

The accompanying drawings, which are included as part of the detailed description to help understanding of the present disclosure, provide embodiments of the present disclosure and describe the contents of the present disclosure together with the detailed description, in which:

FIG. 1 is a schematic diagram illustrating a process of processing surgical information proposed by various embodiments of the present disclosure;

FIG. 2 is a flowchart illustrating a method of processing surgical information based on artificial intelligence according to one embodiment of the present disclosure;

FIG. 3 is a flowchart illustrating in more detail a process of generating a composite feature vector in the method of processing surgical information based on artificial intelligence in FIG. 2 according to one embodiment of the present disclosure;

FIG. 4 is a block diagram illustrating the overall structure for surgical information processing according to one embodiment of the present disclosure;

FIG. 5 is a block diagram illustrating a multi-modal fusion block (MMFB) for fusing feature information;

FIG. 6 is a block diagram illustrating a selective gate block (SGB) for integrating feature information in a balanced manner;

FIG. 7 is a diagram illustrating an iterative refinement process for detecting instruments;

FIG. 8 is a block diagram illustrating a data processing process of the iterative refinement process in FIG. 7;

FIG. 9 is a flowchart illustrating a method of processing surgical information based on artificial intelligence according to another embodiment of the present disclosure;

FIG. 10 is a diagram illustrating a process of classifying surgical procedures in the method of processing surgical information based on artificial intelligence in FIG. 9 according to another embodiment of the present disclosure;

FIG. 11 is a diagram illustrating a process of predicting a next surgical procedure in the method of processing surgical information based on artificial intelligence in FIG. 9 according to another embodiment of the present disclosure;

FIG. 12 is a flowchart illustrating a method of processing surgical information based on artificial intelligence according to still another embodiment of the present disclosure;

FIG. 13 is a diagram illustrating a process of providing medical assistance information to medical staff using the methods of processing surgical information according to various embodiments of the present disclosure; and

FIG. 14 is a block diagram illustrating an apparatus for processing task information based on artificial intelligence according to one embodiment of the present disclosure.

### DETAILED DESCRIPTION

Before describing various embodiments of the present disclosure, in performing image processing in the medical field, a multi-modal data processing method that uses both image input and text input will be described in more detail. In relation to medical images, hereinafter, the term “surgery” is used, but this term should be construed as encompassing all simple treatments or procedures performed by medical staff in addition to surgery. Furthermore, as long as the technical features proposed by various embodiments of the present disclosure are maintained, this term may be extended and applied to various “task” operations beyond “surgical” operations limited to the medical field. For example, target images may include images including instruments associated with household activities in a home environment, or images including instruments associated with manufacturing processes in a manufacturing factory environment. Accordingly, the term “surgery” may be replaced with the term “task” in the present disclosure.

To implement surgical instrument segmentation based on multi-modal data processing, vision-language models may be considered. The vision-language models may use text-promptable segmentation.

This model generates masks based on text descriptions, but has a problem that it is assumed that the described object is present in the image. Accordingly, this assumption can lead to incorrect masks when the object is not actually present. In addition, the same problem may occur in text-promptable surgical instrument segmentation. This method may use a large language model (e.g., GPT-4) to generate segmentation masks based on text descriptions that include the name and additional attributes of instruments. However, this process also assumes that the described instrument is necessarily present, resulting in incorrect masks when the instrument is not present.

As described above, the conventional text-based surgical instrument segmentation methods always assume that the described instrument is present in the image, leading to incorrect masks when the instrument is not actually present. To solve this problem, a method of first detecting an instrument in an image and then generating a prompt for the corresponding instrument may be used. As a result, such an approach selectively uses a prompt only for an object detected in the image, leading to the problem of using information inaccessible to vision-based models.

Meanwhile, referring image segmentation (RIS) segments an object based on given textual representations. A major limitation of conventional reference image segmentation models is that they assume that all textual representations necessarily correspond to objects in the image. Accordingly, an incorrect mask can be generated when the corresponding object is not actually present.

Various embodiments of the present disclosure to be presented below are devised to solve the above problems and are intended to provide the technical configuration capable of generating a segmentation map for all categories even under robust conditions by receiving both image and text and using textual prompts for all classes of objects presented through text input, regardless of their presence in the image.

Hereinafter, embodiments of the present disclosure will be described in detail with reference to the drawings. However, the detailed descriptions of known functions or components that can obscure the gist of the embodiments in the following descriptions and the accompanying drawings will be omitted. In addition, throughout the specification, when a certain portion “includes” a certain component, it means that the certain portion may further include the other component rather than precluding the other component unless specifically stated to the contrary.

In addition, terms such as “first,” “second,” and the like may be used to describe various components, but the components should not be limited by the terms. The terms may be used to distinguish one component from another component. For example, a first component may be referred to as a second component, and similarly, the second component may also be referred to as the first component without departing from the scope of the present disclosure.

The terms used in the present disclosure are only used to describe specific embodiments and are not intended to limit the present disclosure. The singular includes the plural unless the context clearly dictates otherwise. In the present disclosure, it should be understood that the term “include” or “have” is intended to specify that a feature, a number, a step, an operation, a component, a part, or a combination thereof is present, but does not preclude the possibility of the presence or addition of one or more other features, numbers, steps, operations, components, parts, or combinations thereof in advance.

Unless especially defined otherwise, all terms used herein, including technical or scientific terms, have the same meaning as commonly understood by those skilled in the art to which the present disclosure pertains. The terms defined in a generally used dictionary should be construed as having meanings that coincide with the meanings of the terms from the context of the related technology and are not construed as an ideal or excessively formal meaning unless clearly defined in the present disclosure.

According to various embodiments of the present disclosure, text prompts for all classes in a dataset are systematically integrated regardless of whether an object is present in an image, thereby enabling a model to dynamically infer the presence of the object. In particular, various embodiments of the present disclosure include both negative prompts for non-existent objects and positive prompts for existing objects, thereby eliminating such bias and enabling the model to proactively distinguish the presence of objects during a segmentation process. Accordingly, false positive issues occurring in conventional text-prompt-based surgical instrument segmentation models can be effectively resolved, and harmony between vision-based segmentation models and prompt-based models can be achieved.

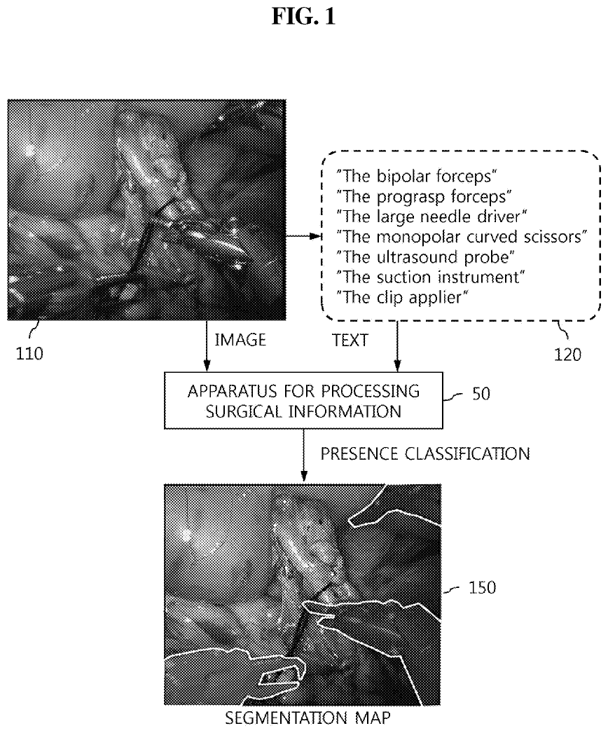

FIG. 1 is a schematic diagram illustrating a process of processing surgical information proposed by various embodiments of the present disclosure. An apparatus 50 for processing surgical information receives a surgical image 110 and surgical-related text 120, detects instruments present in the surgical image 110, and outputs a segmentation map 150. In this case, the text may consist of values provided regardless of whether objects are present in the image, and for example, may include relevant attributes, such as the name or appearance of a surgical instrument. That is, the text prompt may include objects of a class present in the image, or objects of a class that is not present in the image. Based on this text input, the apparatus 50 for processing surgical information may classify whether objects of a given class are present in the image, generates masks for objects corresponding to the classified class, and outputs a segmentation map.

For convenience of description, the problem to be solved by various embodiments, which will be presented below, is defined using equations as follows.

Surgical instrument segmentation (SIS) is the task of segmenting surgical instruments from an endoscopic surgical image I∈ [symbol] ^{H×W×3}. Here, H and W denote a height and width of the image, respectively. In text prompt-based surgical instrument segmentation, an image I is combined with a text prompt T including the name or attribute of a surgical instrument, allowing the segmentation task to respond to a specific prompt.

In traditional vision-based surgical instrument segmentation, the model receives only the image I as input and predicts a mask M∈ [symbol] ^{H×W×C} including C classes, each of which corresponds to a specific surgical instrument. However, in the text prompt-based surgical instrument segmentation, the image I and a class-specific text prompt t_{c} are provided as input for each class c. Accordingly, the model may generate a binary mask M_{c}∈ [symbol] ^{H×W×1} for each class c.

In the robust text prompt-based surgical instrument segmentation proposed by various embodiments of the present disclosure, a presence probability p_{c} is generated to classify the presence of class c within the image for the text prompt t_{c}.

In a test stage, inference is performed for all C classes for the given image I, obtaining class-specific masks M₁, . . . , M_{C} and presence probabilities p₁, . . . , p_{C}. This output is aggregated into a final mask M∈ [symbol] ^{H×W×C}, and each pixel in mask M represents class-specific segmentation.

To ensure the robust text prompt-based surgical instrument segmentation, the presence probabilities may be incorporated so that the model may first assess the presence of each instrument class. This setting reduces false positives and improves segmentation accuracy by avoiding generating unnecessary masks for non-existent classes.

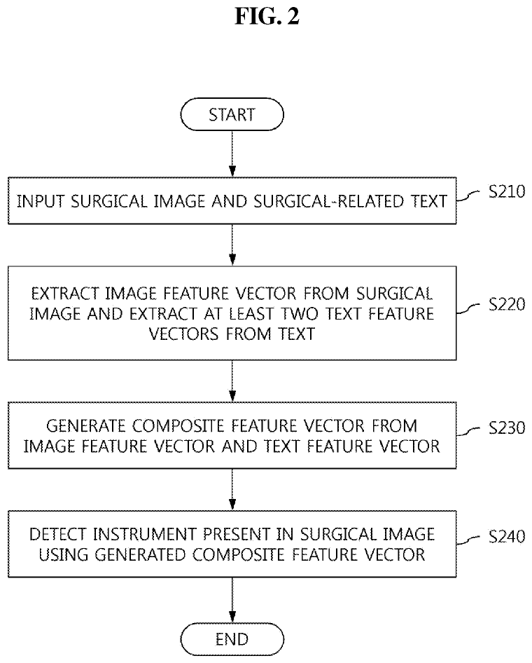

FIG. 2 is a flowchart illustrating a method of processing surgical information based on artificial intelligence according to one embodiment of the present disclosure and illustrates a series of processing operations performed by the apparatus for processing surgical information, which includes at least one processor.

In operation S210, the apparatus for processing surgical information receives a surgical image and surgical-related text. Here, the surgical-related text may include the name or attributes of the surgical instrument, and the surgical instrument corresponds to a class of an object to be detected thereafter. This text may be input into a large language model by prompts.

In operation S220, the apparatus for processing surgical information extracts an image feature vector from the received surgical image and at least two text feature vectors from the received text. Various embodiments of the present disclosure propose an iterative refinement strategy for multi-modal input to improve segmentation accuracy. In a first stage of the iterative refinement process, initial segmentation is performed based on prompts using instrument names and a large language model. Next, in a second stage, the segmentation results are refined using location-based prompts derived from the initial prediction results. Accordingly, the process of extracting image feature vectors is performed for a single surgical image, while the process of extracting text feature vectors is performed through a sequential two-step iterative process. Since different text inputs are repeated, at least two text feature vectors may be extracted.

Regarding the process of generating prompts for extracting feature vectors, various methods of generating a text prompt the for each class c may be provided. In a first iteration according to one embodiment of the present disclosure, prompts are generated using class names, for example, in a format such as “The {cls name}”. In addition, GPT-4 is used to generate detailed prompts describing the visual characteristics of each class, which are maintained during training and testing stages. This combination may provide both general and specific prompts, enabling the model to recognize surgical instruments based on their names and appearances.

In the second iteration according to one embodiment of the present disclosure, since surgical instruments often appear in consistent locations on screen during surgery, the prompts are further enhanced with location-based descriptions. To this end, the centroid of an actual mask of each instrument is calculated to identify the general location of the corresponding instrument, and a prompt such as “The {cls name} on the {location}” is generated. For example, a location prompt may categorize a location into four options: “left-top,” “left-bottom,” “right-top,” and “right-bottom.” During model training, for each class c, all three types of text prompts, that is, class name prompts, GPT-4 visual prompts, and location prompts, are used to provide comprehensive contextual information.

When the model is trained solely by positive prompts that describe only objects present in the image, the model may develop a bias toward the presence of instruments, thereby reducing generalization ability. To prevent this, various embodiments of the present disclosure may generate negative prompts for classes that are not present in the image, thereby improving the classification ability to distinguish between existing and non-existent classes. For classes that are not present in the image, negative examples are sufficiently provided using the class name, GPT-4 description prompts, and location prompts randomly selected from four options. Accordingly, stable training can be secured by balancing the positive and negative prompts corresponding to a single image.

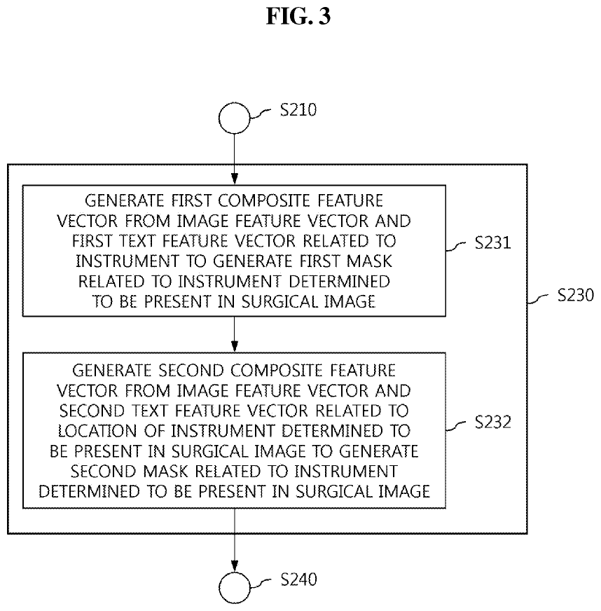

FIG. 3 is a flowchart illustrating in more detail a process of generating a composite feature vector (S230) in the method of processing surgical information in FIG. 2 according to one embodiment of the present disclosure.

In operation S231, the apparatus for processing surgical information may generate a first composite feature vector from an image feature vector and a first text feature vector related to the instrument, thereby generating a first mask for the instrument determined to be present in the surgical image. To this end, the first text feature vector extracted based on at least one text input related to the name or attribute of the instrument and the image feature vector are multi-modally fused to generate the first composite feature vector. Then, a segmentation mask may be generated corresponding to an instrument whose probability of presence in the surgical image is greater than or equal to a first reference value among instruments input via text by referencing the generated first composite feature vector.

In operation S232, the apparatus for processing surgical information may generate a second composite feature vector from the image feature vector and a second text feature vector regarding the location of the instrument determined to be present in the surgical image, thereby generating a second mask for the instrument determined to be present in the surgical image. To this end, the second text feature vector extracted based on text input regarding the location of the instrument derived using the first mask and the image feature vector are multi-modally fused to generate the second composite feature vector. Then, a segmentation mask may be generated corresponding to an instrument whose probability of presence in the surgical image is greater than or equal to a second reference value among instruments input via text by referencing the generated second composite feature vector.

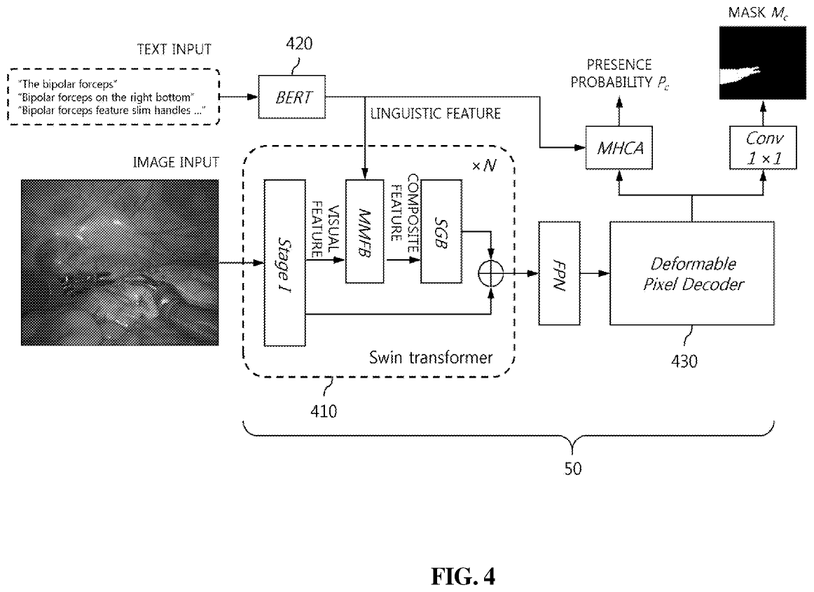

Returning back to FIG. 2, in operation S230, the surgical information processing device generates a composite feature vector from the image feature vector and the text feature vector. The surgical instrument segmentation method according to one embodiment of the present disclosure includes two core modules based on robust conditions. First, a multi-modal fusion block (MMFB) effectively integrates visual and text features. Second, a selective gate block (SGB) dynamically balances the visual information and the fused features. A more detailed configuration will be described below with reference to FIGS. 4 to 6.

In operation S240, the apparatus for processing surgical information detects instruments present in the surgical image using the generated composite feature vector. During this process, the generated composite feature vector fuses a plurality of masks generated for instruments determined to be present in the surgical image, thereby generating a single mask. Then, based on the calculated single mask, a segmentation map for the instruments present in the surgical image is output. To obtain the single mask, various fusion methods may be used, such as calculating the average of the plurality of masks, calculating a weighted sum of the plurality of masks, or selecting a maximum value among the plurality of masks.

FIG. 4 is a block diagram illustrating the overall structure for processing surgical information according to one embodiment of the present disclosure, and the structure of the apparatus 50 for processing surgical information is designed using an encoder-decoder structure to achieve robust text prompt-based surgical instrument segmentation. In particular, a binary classifier is added in parallel with a decoder 430 to determine the presence of instruments.

First, an image I and a text description T are encoded using an image encoder and a text encoder, respectively. The image encoder is based on, for example, a Swin Transformer 410, which is specialized for a dense prediction task. The text encoder uses a BERT 420. The visual features extracted at stage i of the Swin Transformer 410 are denoted by vᵢ∈ [symbol] ^{H}^{ᵢ}^{×W}^{ᵢ}^{×C}^{ᵢ}, and the linguistic features extracted by the BERT 420 are denoted by l∈ [symbol] ^{T×C}^{ₗ}. Here, Hᵢ and Wᵢ denote the height and width of the visual features, T denotes the number of words in the text description, and Cᵢ and Cₗ denote the number of channels for the visual and linguistic features, respectively.

### Encoder Design

Various embodiments of the present disclosure introduce early feature fusion to efficiently integrate vision and linguistic knowledge, and the goal of early fusion is to enhance semantic understanding and improve feature interactions. To this end, in the embodiment of FIG. 4, the MMFB, which effectively integrates the visual and textual features and the SGB, which dynamically balances the visual information and the fused features, are added between the stages of the Swin Transformer 410.

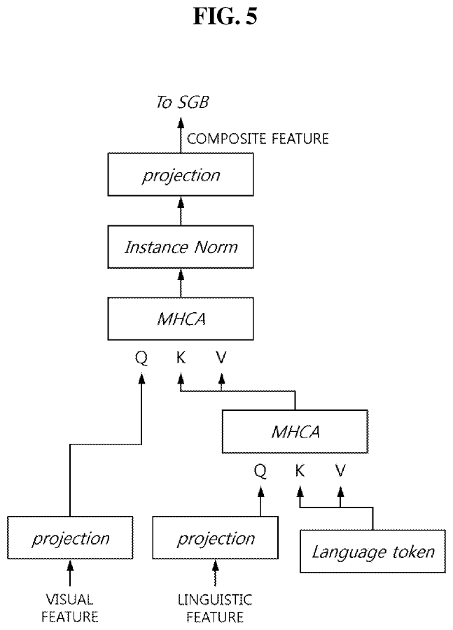

FIG. 5 is a block diagram illustrating the MMFB for fusing feature information. The MMFB is designed to integrate vision and language features through a multi-stage fusion process, and the fused features are then delivered to a gating module (SGB).

The fusion process begins by independently projecting visual and language features into a compatible feature space. The MMFB includes two multi-head cross attention (MHCA) modules. A first MHCA uses language tokens to fuse with language features. The language tokens are learnable parameters that adjust language features before fusion. Since the decoder according to one embodiment of the present disclosure does not use the language features generated during fusion, the decoder focuses on selecting only the information necessary for fusing with visual features. Accordingly, the first MHCA finely tunes the language features, and a second MHCA is configured to interact with the visual features.

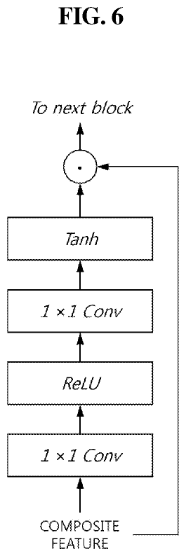

FIG. 6 is a block diagram illustrating the SGB for integrating feature information in a balanced manner. The SGB is introduced after the MMFB to adjust the flow of fused features and prevent language features from dominating the visual information. The SGB dynamically controls the importance of features, allowing the model to maintain a balanced representation of visual and language features and processes this information before delivering it to the next stage. As illustrated in FIG. 6, the SGB receives the fused features as input, performs several light transformations, and applies a gating mechanism.

The overall fusion process between the stages may be represented by the following equation.

$${v}_{i} = {V}_{i} ( {f}_{i - 1} ) , i ∈ \{ 1 , 2 , 3 , 4 , \} \qquad [ Equation 1 ]$$

$${f}_{i} = \{ \begin{matrix}{v}_{i} + {SGB}_{i} ( {MMFB}_{i} ( {v}_{i} , l ) ) , i ∈ \{ 1 , 2 , 3 , 4 \} , \\ I , i = 0\end{matrix} \qquad [ Equation 2 ]$$

Here, Vᵢ denotes an i^{th} stage of the Swin Transformer, and SGBᵢ and MMFBᵢ denote an i^{th} fusion block located between Vᵢ and Vᵢ₊₁.

### Decoder Design

The decoder proposed in various embodiments of the present disclosure is designed to perform two tasks: classifying the presence of an object designated by a text prompt and generating a corresponding segmentation mask. To this end, one embodiment of the present disclosure uses, for example, a multi-scale deformable attention pixel decoder. This decoder effectively uses multi-scale features by integrating a feature pyramid network structure and receives f₁, f₂, f₃, and f₄ as inputs. A final segmentation mask M_{c} is generated by allowing the output of the decoder to pass through a 1×1 convolution layer.

In addition to generating the mask, the embodiment of FIG. 4 introduces a parallel branch for calculating a presence probability of an object described by a text prompt. This branch fuses the output of the decoder from f₄ with raw language features extracted from the BERT using the MHCA. This fusion allows the decoder to use both visual and textual information, thereby enhancing its ability to determine the presence of a designated object. The embodiment of FIG. 4 directly uses the raw language features extracted from the BERT for presence prediction, rather than using the fused language features from the encoder. Accordingly, a richer semantic context can be maintained. After fusion using the MHCA, the presence probability p_{c} is calculated by allowing the output to pass through a linear layer.

Finally, the embodiment of FIG. 4 calculates the cross-entropy loss for the generated mask and presence probability, allowing the model to optimize the accuracy in segmentation and presence detection tasks. This may be represented by the following Equation 3.

$$L = BCELoss ( {p}_{c} , {y}_{c} ) + λ \cdot CELoss ( {M}_{c} , {M}_{c}^{gt} ) \qquad [ Equation 3 ]$$

Here, λ denotes a hyperparameter for the mask loss, and y_{c} and

$${M}_{c}^{gt}$$

denote the ground truth for presence and the mask of class c, respectively.

### Iterative Refinement

Various embodiments of the present disclosure propose an iterative refinement strategy that uses the location prompts used in training during inference. In the training stage, the location prompts may be generated because the ground truth of the instrument is accessible, but in the inference stage, the location prompts may not be generated. Accordingly, the inference process is performed through two iterations.

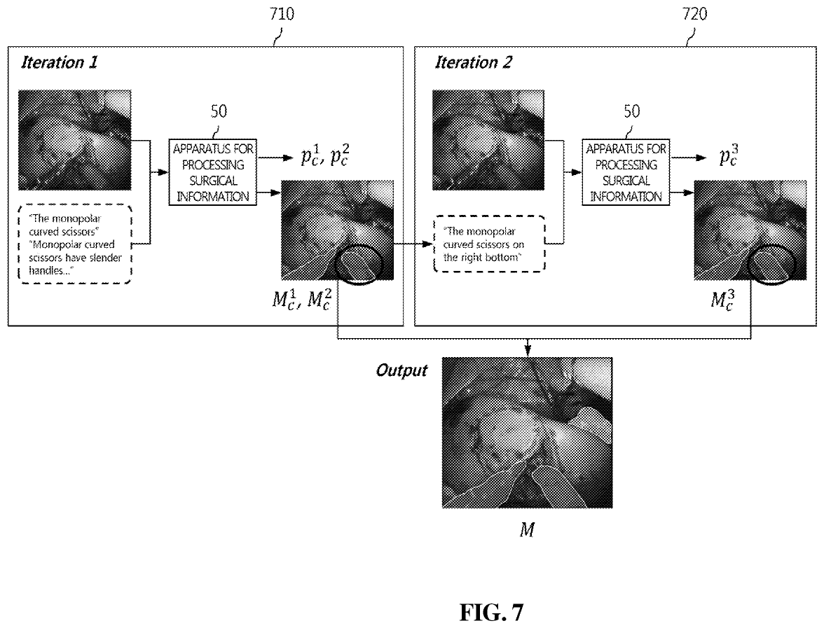

FIG. 7 is a diagram illustrating an iterative refinement process for detecting instruments.

In a first iteration 710, the apparatus 50 for processing surgical information generates an initial segmentation map and presence probability for each instrument category. To this end, two types of prompts are used: a category name prompt (e.g., “The monopolar curved scissors”) and a descriptive prompt generated by GPT-4 to provide visual characteristics of the instrument. For category c, the model proposed in the present embodiment predicts segmentation maps

$${M}_{c}^{1} and {M}_{c}^{2} ,$$

and presence probabilities

$${p}_{c}^{1} and {p}_{c}^{2} .$$

When it is determined that an average presence probability

$$( {p}_{c}^{1} + {p}_{c}^{2} ) / 2$$

exceeds a reference value (e.g., 0.5) and class c is present in the image, the process proceeds to a second iteration 720.

In the second iteration 720, the apparatus 50 for processing surgical information refines segmentation using spatial information. Based on the segmentation maps

$${M}_{c}^{1} and {M}_{c}^{2}$$

obtained in the first iteration 710, the model proposed in the present embodiment may calculate a centroid of a detected object and determine an approximate location of the object within one of four quadrants, namely an upper-left quadrant, a lower-left quadrant, an upper-right quadrant, or a lower-right quadrant. This location information is integrated into a new location prompt (e.g., “The monopolar curved scissors on the right bottom”) and used as input for the second inference. The model proposed in the present embodiment generates a segmentation map

$${M}_{c}^{3}$$

and an existence probability

$${p}_{c}^{3}$$

for class c.

For the final output M_{c}, the segmentation maps generated in two iterations 710 and 720 may be combined as shown in the following Equation 4.

$${M}_{c} = \{ \begin{matrix}0 , & if \frac{{p}_{c}^{1} + {p}_{c}^{2}}{2} < 0.5 \\ \frac{{M}_{c}^{1} + {M}_{c}^{2}}{2} , & \frac{{p}_{c}^{1} + {p}_{c}^{2}}{2} \geq 0.5 \& {p}_{c}^{3} < 0.5 \\ \frac{{M}_{c}^{1} + {M}_{c}^{2} + {M}_{c}^{3}}{3} , & \frac{{p}_{c}^{1} + {p}_{c}^{2}}{2} \geq 0.5 \& {p}_{c}^{3} \geq 0.5\end{matrix} \qquad [ Equation 4 ]$$

This combination uses both fixed and location prompts to generate a more precise and context-sensitive segmentation map. This iterative refinement approach can improve segmentation performance in complex surgical images and ensure robust segmentation by gradually refining predictions based on text and spatial prompts.

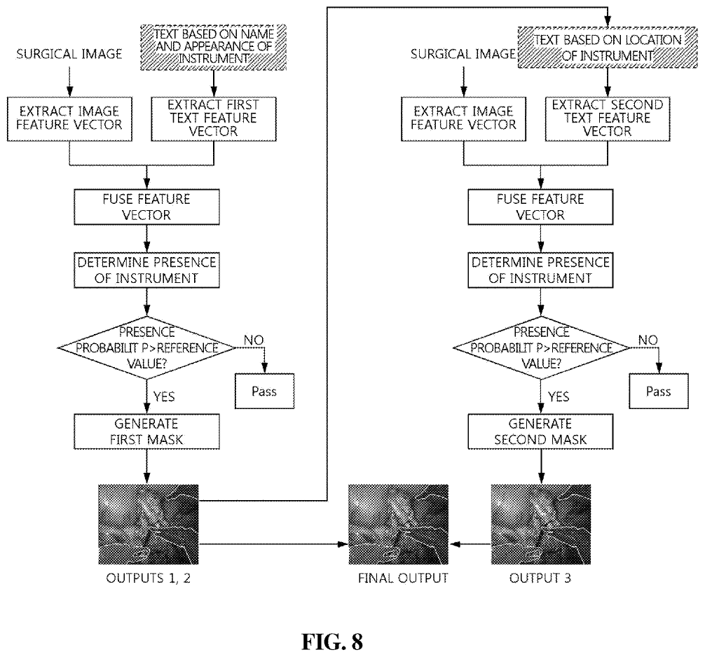

FIG. 8 is a block diagram illustrating a data processing process of the iterative refinement process in FIG. 7.

Feature vector extraction, synthesis, and mask generation used in the left process (first iteration) and the right process (second iteration) may be performed by sharing the same model. Based on the masks obtained in the first iteration, the locations of the instruments are divided into four categories (top left, bottom left, top right, bottom right) to generate the text input for the second iteration. Each mask is represented by a probability value for the presence of the instrument provided as a text input in pixels and may be output as binary data. The final output may be calculated by fusing three masks based on the presence probability p of the instrument. As described above, to obtain the single mask, various fusion methods may be used, such as calculating the average of the plurality of masks, calculating a weighted sum of the plurality of masks, or selecting a maximum value among the plurality of masks. Equation 4 exemplifies a method of generating a single mask using the average. The reason why there are three masks illustrated in FIG. 8 is that two masks are generated in the first iteration using two text inputs (name and appearance of an instrument), and one mask is generated in the second iteration using one text input regarding the location of the instrument, resulting in a total of three masks.

As described above, in the method of processing surgical information according to one embodiment of the present disclosure, when generating a composite feature vector, a first composite feature vector is generated from an image feature vector and a first text feature vector regarding the instrument, thereby generating a first mask for the instrument determined to be present in the surgical image. From an implementation perspective, this process independently projects the image feature vector representing visual features and the first text feature vector representing linguistic features based on a prompt input indicating the name or attribute of the instrument into a feature space through the encoder, and generates the first composite feature vector using the MHCA. In addition, the decoder may reference the first composite feature vector to calculate the presence probability for each class of all objects designated by the text input, and generate the first mask representing the initial segmentation region for objects with a presence probability greater than or equal to the first reference value.

Then, a second composite feature vector is generated from the image feature vector and the second text feature vector relating to the location of the instrument determined to be present in the surgical image, thereby generating a second mask for the instrument determined to be present in the surgical image. From an implementation perspective, this process determines the location of the instrument within the image for the object detected from the first mask through the encoder, independently projects the image feature vector representing visual features and the second text feature vector representing linguistic features based on the prompt input indicating the location of the instrument into a feature space, and generates the second composite feature vector using the MHCA. In addition, the decoder may reference the second composite feature vector to calculate the presence probability for each class of objects located based on the text input, and generate the second mask representing a refined segmentation region for objects with a presence probability greater than or equal to the second reference value.

Through the iterative refinement process of FIGS. 7 and 8, various embodiments of the present disclosure use both image and text data simultaneously, enabling the detection of instruments not present during training, unlike models that use only image data. In addition, when using both image and text data simultaneously, the presence of an instrument is first determined and then a mask is generated, which is highly effective in preventing false positives. In particular, detection performance can be improved using various text inputs (attribute information such as the name and appearance of an instrument, instrument location information), and precise instrument detection based on the location of the instrument is also possible through two iterations.

Meanwhile, one embodiment of the present disclosure may further include a separate detection algorithm to improve the detection performance of small, detailed objects, such as a thread and a needle. For a thread and a needle, unlike other instruments, a separate structure is employed in which a thick region in an image is first inferred, and a more accurate region of the thread and the needle is then inferred through a refinement network that receives, as inputs, mask information of the corresponding region and an image in which the inferred region is removed and a background is filled. Therefore, it is possible to develop a separate detection algorithm for small, detailed objects and used it in conjunction with the surgical instrument segmentation model.

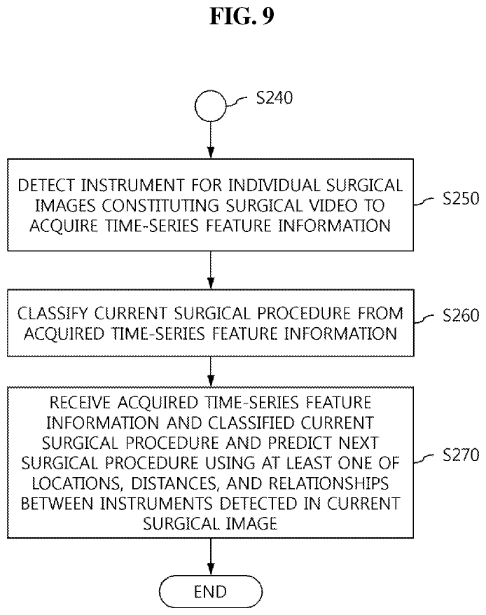

FIG. 9 is a flowchart illustrating a method of processing surgical information based on artificial intelligence according to another embodiment of the present disclosure and proposes an additional processing process that may be performed sequentially following the method of processing surgical information of FIG. 2.

In operation S250, the apparatus for processing surgical information extracts feature vectors from individual surgical images constituting the surgical video to acquire time-series feature information. Unlike instrument detection performed on a single surgical image, this process uses video input to perform image recognition and prediction based on a model that combines spatial and visual information within the image with time-series information obtained from the video. In addition, in addition to simply detecting the instrument, the apparatus acquires features related to the surgical action being performed within the image, including information about the object corresponding to the instrument or the movement using the instrument, for use as analysis material in subsequent operations.

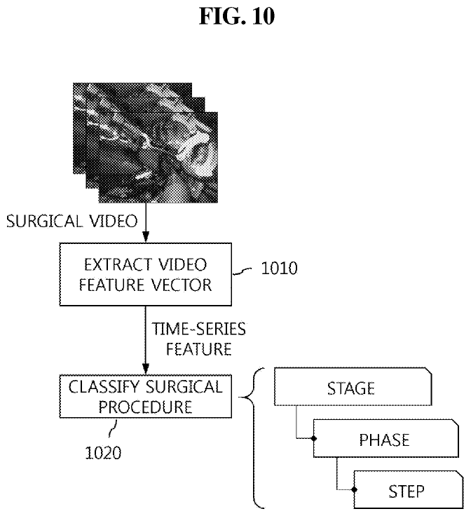

In operation S260, the apparatus for processing surgical information may classify the current surgical procedure based on the acquired time-series feature information. Referring to FIG. 10, a process of classifying a current surgical procedure may output (1020), as a classification result, a surgical procedure having a highest probability value from the time-series feature information using at least one hierarchical classifier based on a fusion model of spatial visual information and temporal flow information in an image acquired through a video feature vector extraction process 1010. Here, the surgical procedure follows a hierarchical structure of a stage, a phase, and a step according to a classification granularity, and may be classified using a single hierarchy or a combination of two or more hierarchies. For example, it is possible to recognize and/or predict only a current step, to recognize and/or predict a combination of a current phase and step, or to recognize and/or predict a combination of a current stage, phase, and step. Accordingly, for example, when a user desires to classify the current surgical procedure as a combination of a phase and a step, a phase step combination corresponding to the highest probability values for the phase and t e step may be output.

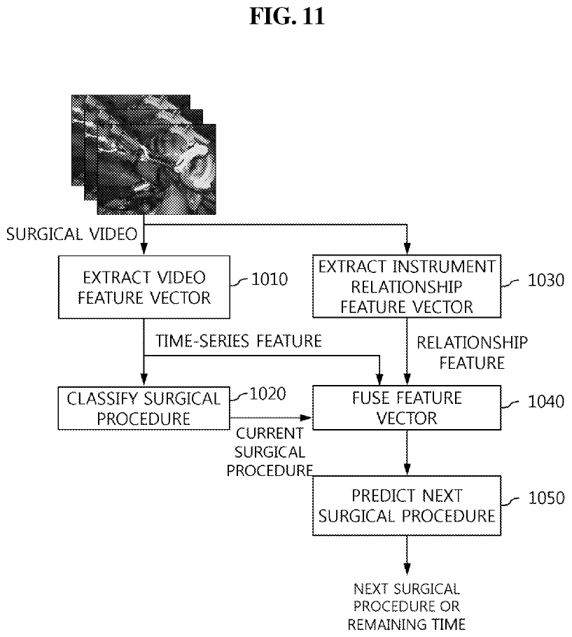

Referring back to FIG. 9, in operation 0S270, the apparatus for processing surgical information may receive the acquired time-series feature information and the classified current surgical procedure, and predict a next surgical procedure using at least one of positions, distances, and interrelationships among instruments detected in the current surgical image. Referring to FIG. 11, a process of predicting a next surgical procedure may reference the fusion model (1040) to determine a surgical procedure having a highest probability of appearing temporally consecutively to the current surgical procedure as the next surgical procedure (1050), and may output at least one of the determined next surgical procedure or a remaining time until the next surgical procedure. In this case, the time-series features acquired through the video feature vector extraction process (1010), the current surgical procedure acquired through the surgical procedure classification (1020), and the interrelationship features acquired through an instrument interrelationship feature vector extraction process (1030) may all be referred to in the feature vector fusion process (1040).

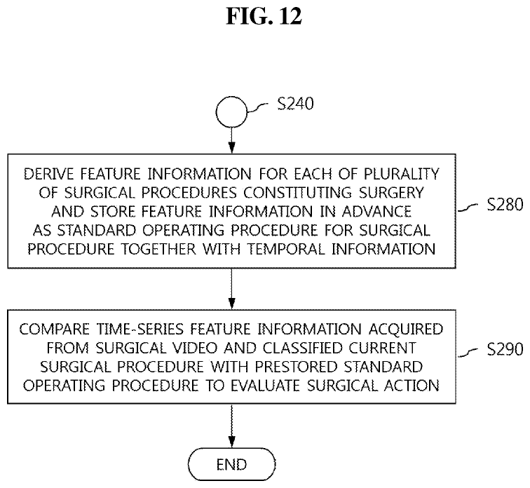

FIG. 12 is a flowchart illustrating a method of processing surgical information based on artificial intelligence according to still another embodiment of the present disclosure and proposes an additional processing process that may be performed sequentially following the method of processing surgical information of FIG. 2.

In operation S280, the apparatus for processing surgical information derives feature information for each of a plurality of surgical procedures constituting a surgery and stores the feature information together with temporal information in advance as a standard operating procedure for the surgical procedure. Here, the standard operating procedure is constructed by acquiring feature information of exemplary surgical procedures and building a ground-truth database in advance, and is used as a comparison reference when a new surgical image or surgical video is subsequently input.

In operation S290, the apparatus for processing surgical information may compare the time-series feature information acquired from the surgical video and the classified current surgical procedure with the pre-stored standard operating procedure to evaluate at least one of precision, skill, completion, efficiency, and safety of a surgery. Here, the precision is an evaluation metric indicating surgical accuracy and error tolerance, the skill is an evaluation metric indicating technical proficiency and dexterity, the completion is an evaluation metric indicating completeness of a surgical outcome, the efficiency is an evaluation metric indicating time and resource utilization, and the safety is an evaluation metric indicating patient protection and complication prevention. For example, a surgical situation may be recognized to predict a surgical experience level of a currently performing surgeon, or evaluation items for surgical actions may be quantified as scores.

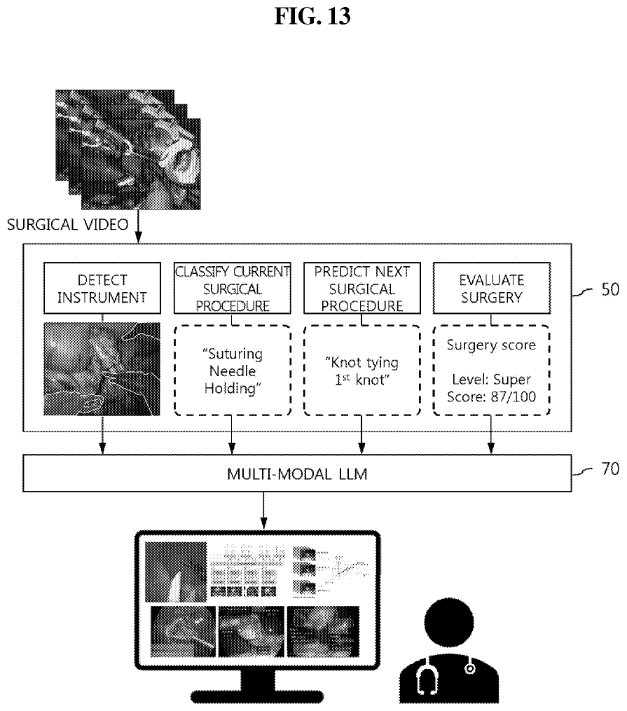

FIG. 13 is a diagram illustrating a process of providing medical assistance information to medical staff using the methods of processing surgical information according to various embodiments of the present disclosure.

The apparatus 50 for processing surgical information may receive an actual surgical scene, detect instruments, classify a current surgical procedure, or predict a next surgical procedure based on the current surgical procedure. In addition, the apparatus may analyze a surgical procedure in real time and provide evaluation scores for surgical actions.

The information generated in this way is input to a multi-modal large language model 70, which performs an integrated analysis of an overall surgical situation and then provides analysis results or surgical assistance information to medical staff. For example, when analysis results are visually displayed on a screen through a graphical interface, medical staff may refer to the AI analysis results in real time while performing a surgery. To this end, a display device may be used as an output unit, and a headset or a head-mounted display (HMD) employing extended reality (XR) technologies, including virtual reality, augmented reality, and mixed reality, may be adopted to display analysis results or surgical assistance information together with surgical images.

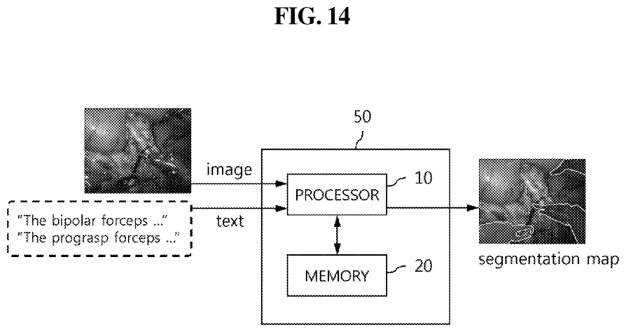

FIG. 14 is a block diagram illustrating an apparatus for processing task information based on artificial intelligence according to one embodiment of the present disclosure, which reconstructs the method of processing surgical information of FIG. 2 from a hardware configuration perspective and extends the medical-domain “surgery” to a more general “task” domain. Accordingly, to avoid the overlapping descriptions, descriptions will be briefly provided focusing on functions and operations of each component. Regarding the above-described specific configuration, terms such as surgical images and surgical instruments are replaced with task images and task instruments, respectively. For example, the apparatus for processing task information according to the present embodiment may detect task instruments such as knives or cutting boards from images of cooking in a home environment, or may process task-related information related to cooking.

The apparatus 50 for processing task information includes a memory 20 for storing a program for processing task information, and a processor 10 for executing the program stored in the memory 20. The program may receive task images and task-related text as inputs, extract image feature vectors from the task images, extract at least two text feature vectors from the text, generate a composite feature vector from the image feature vectors and the text feature vectors, and detect instruments present in the task image using the generated composite feature vector.

In the apparatus 50 for processing task information, the program may execute instructions to generate the composite feature vector by generating a first composite feature vector from the image feature vector and a first text feature vector related to an instrument, generating a first mask for an instrument determined to be present in the task image, generating a second composite feature vector from the image feature vector and a second text feature vector related to a location of the instrument determined to be present in the task image, and generating a second mask for the instrument determined to be present in the task image.

The program may execute instructions to generate the first mask by generating the first composite feature vector by performing multi-modal fusion of the image feature vector and the first text feature vector extracted based on at least one text input related to a name or an attribute of an instrument and generating a segmentation mask corresponding to an instrument having a probability of being present in the task image greater than or equal to a first reference value among instruments input via text by referencing the generated first composite feature vector.

In addition, the program may execute instructions to generate the second mask by generating the second composite feature vector by performing multi-modal fusion of the second text feature vector extracted based on a text input related to a location of the instrument derived using the first mask and the image feature vector and generating a segmentation mask corresponding to an instrument having a probability of being present in the task image greater than or equal to a second reference value among instruments input via text by referencing the generated second composite feature vector.

In the apparatus 50 for processing task information, the program may perform instructions to detect an instrument present in the task information by generating a single mask by fusing a plurality of masks generated for an instrument determined to be present in the task image using the generated composite feature vector and outputting a segmentation map for the instrument present in the task image based on the generated single mask.

In the apparatus 50 for processing task information, the program may further include instructions to extract feature vectors from individual task images constituting a task video to acquire time-series feature information and classify a current task process based on the acquired time-series feature information.

In addition, the program may further include instructions to receive the acquired time-series feature information and the classified current task process and predict a next task process using at least one of locations, distances, and relationships among instruments detected in the current task image.

Various embodiments of the present disclosure may be implemented by various means, such as hardware, firmware, software, or a combination thereof. When implemented by hardware, one embodiment of the present disclosure may be implemented by one or more application specific integrated circuits (ASICs), digital signal processors (DSPs), digital signal processing devices (DSPDs), programmable logic devices (PLDs), field programmable gate arrays, controllers, microcontrollers, microprocessors, etc. In the case of implementation through firmware or software, an embodiment of the present disclosure may be implemented in the form of modules, procedures, functions, or the like configured to perform the capabilities or operations described above. The software code may be stored in a memory and executed by a processor. The memory may be located inside or outside the processor and may exchange data with the processor through various known means.

Meanwhile, various embodiments of the present disclosure may be implemented with computer-readable code on a computer-readable recording medium. The computer-readable recording media include all types of recording devices that store data readable by a computer system. Examples of the computer-readable recording media include a ROM, a RAM, a CD-ROM, a magnetic tape, a floppy disk, optical data storage devices, etc. In addition, the computer-readable recording media may be distributed across a network-connected computer system to allow the computer-readable code to be stored and executed in a distributed manner. In addition, functional programs, codes, and code segments for implementing the present disclosure can be easily inferred by programmers in the art to which the present disclosure pertains.

Accordingly, various embodiments of the present disclosure may be implemented as one or more non-transitory computer-readable media for storing one or more instructions. Here, the one or more instructions executable by one or more processors perform image processing to generate a three-dimensional (3D) model by receiving a two-dimensional (2D) image as input, and receive a text description of regions not visible in the input 2D image to generate a panorama image extended from the input 2D image based on the input text description, and generate a 3D model by generating a 3D object from an object present in the generated panorama image and arranging the 3D object in a 3D space. According to various embodiments of the present disclosure, the panorama image extended from the 2D image may be constructed using the text description and condition data for regions not visible in the 2D image, and the 3D model may be generated based on 3D scene information inferred from the panorama image and a 3D model database, thereby enabling reproduction of high-quality and precise 3D images without requiring specialized hardware or multiple image inputs, and an editable 3D model based on a reference image can be easily constructed, thereby providing an image service with a high degree of freedom.

According to various embodiments of the present disclosure, in implementing the multi-modal based surgical instrument segmentation model that uses both image input and text input, the segmentation map can be generated through prompt-based iterative refinement without prior knowledge of the existence of objects in the image, thereby enabling detection of instruments that did not appear during learning, effectively preventing false positive problems, and enabling precise instrument detection even under robust conditions by balancing the vision-based model and the prompt-based model. Accordingly, it is possible to minimize the risk of accidents that may occur during image-based surgical procedures performed by medical staff in environments with limited visibility, and by providing quantitative analysis results for surgical procedures through surgical quality evaluation, the surgical competency of medical staff may be objectively assessed or used as educational material. Furthermore, by integrating various data derived during surgical procedures and providing intuitive information in real time through a graphical interface, efficient surgical assistance for medical staff can be achieved.

The present disclosure has been described above with reference to various embodiments thereof. Those skilled in the art to which the present disclosure pertains will be able to understand that various embodiments may be implemented in a modified form without departing from the essential characteristics of the present disclosure. Accordingly, the disclosed embodiments should be considered in an illustrative rather than a limiting sense. The scope of the present disclosure is described in the claims rather than the above description, and all differences in the equivalent scope should be construed as being included in the present disclosure.

### DESCRIPTION OF REFERENCE NUMERALS

10: processor
20: memory
50: apparatus for processing surgical (task) information
70: multi-modal large language model

What is claimed is:

1. A method of processing, by an apparatus for processing surgical information including at least one processor, surgical information, the method comprising:
receiving a surgical image and surgical-related text;
extracting an image feature vector from the received surgical image and extracting at least two text feature vectors from the received text;
generating a composite feature vector from the image feature vector and the text feature vector; and
detecting an instrument present in the surgical image using the generated composite feature vector.

2. The method of claim 1, wherein the generating of the composite feature vector includes:
generating a first composite feature vector from the image feature vector and a first text feature vector related to an instrument to generate a first mask for the instrument determined to be present in the surgical image; and
generating a second composite feature vector from the image feature vector and a second text feature vector related to a location of the instrument determined to be present in the surgical image to generate a second mask for the instrument determined to be present in the surgical image.

3. The method of claim 2, wherein the generating of the first mask includes:
generating a first composite feature vector by performing multi-modal fusion of a first text feature vector extracted based on at least one text input related to a name or attribute of an instrument and the image feature vector; and
generating a segmentation mask corresponding to an instrument whose probability of being present in the surgical image is greater than or equal to a first reference value, among instruments input via text by referencing the generated first composite feature vector.

4. The method of claim 3, wherein the generating of the first mask includes:
independently projecting, by an encoder, the image feature vector representing visual features and the first text feature vector representing linguistic features based on a prompt input indicating the name or attribute of the instrument into a feature space and generating the first composite feature vector using multi-head cross attention (MHCA); and
calculating, by a decoder, a presence probability for each class of all objects designated by the text input by referencing the first composite feature vector and generating the first mask representing an initial segmentation region for objects with a presence probability greater than or equal to the first reference value.

5. The method of claim 2, wherein the generating of the second mask includes:
generating a second composite feature vector by performing multi-modal fusion of the second text feature vector extracted based on text input regarding the location of the instrument derived using the first mask and the image feature vector, and
generating a segmentation mask corresponding to an instrument whose probability of being present in the surgical image is greater than or equal to a second reference value, among instruments input via text by referencing the generated second composite feature vector.

6. The method of claim 5, wherein the generating of the second mask includes:
determining, by an encoder, the location of the instrument within the image for the object detected from the first mask, independently projecting the image feature vector representing visual features and the second text feature vector representing linguistic features based on the prompt input indicating the location of the instrument into a feature space, and generating the second composite feature vector using MHCA;
calculating, by a decoder, a presence probability for each class of objects located based on the text input by referencing the second composite feature vector and generating the second mask representing a refined segmentation region for objects with a presence probability greater than or equal to the second reference value.

7. The method of claim 1, wherein the detecting of the instrument present in the surgical image includes:
calculating a single mask by fusing a plurality of masks generated for an instrument determined to be present in the surgical image using the generated composite feature vector, and
outputting a segmentation map for the instrument present in the surgical image based on the calculated single mask.

8. The method of claim 1, further comprising:
acquiring time-series feature information by extracting feature vectors from individual surgical images constituting a surgical video; and
classifying a current surgical procedure from the acquired time-series feature information.

9. The method of claim 8, wherein the surgical procedure follows a hierarchical structure of a stage, a phase, and a step according to a classification granularity and is classified using a single hierarchy or a combination of two or more, and
the classifying of the current surgical procedure includes using at least one hierarchical classifier based on a fusion model of spatial visual information and temporal flow information within the image to output a surgical procedure with a highest probability value from the time-series feature information as a classification result.

10. The method of claim 9, further comprising receiving the acquired time-series feature information and the classified current surgical procedure and predicting a next surgical procedure using at least one of locations, distances, and relationships among instruments detected in a current surgical image.

11. The method of claim 10, wherein the predicting of the next surgical procedure includes referencing the fusion model to determine a surgical procedure having a highest probability of appearing temporally consecutively to the current surgical procedure as the next surgical procedure and outputting at least one of the determined next surgical procedure or a remaining time until the next surgical procedure.

12. The method of claim 8, further comprising:
deriving feature information for each of a plurality of surgical procedures constituting a surgery and storing the feature information together with temporal information in advance as a standard operating procedure for a surgical procedure; and
comparing the time-series feature information acquired from the surgical video and the classified current surgical procedure with the pre-stored standard operating procedure to evaluate at least one of precision, skill, completion, efficiency, and safety of a surgery.

13. A non-transitory computer-readable medium storing one or more instructions for processing surgical information,
wherein the one or more instructions executable by one or more processors comprise:
receiving a surgical image and surgical-related text;
extracting an image feature vector from the received surgical image and extracting at least two text feature vectors from the received text;
generating a composite feature vector from the image feature vector and the text feature vector; and
detecting an instrument present in the surgical image using the generated composite feature vector.

14. An apparatus for processing task information, comprising:
a memory configured to store a program for processing task information; and
a processor configured to execute the program stored in the memory,
wherein the program includes instructions to:
receive a task image and task-related text;
extract an image feature vector from the received task image and extract at least two text feature vectors from the received text;
generate a composite feature vector from the image feature vector and the text feature vector, and
detect an instrument present in the task image using the generated composite feature vector.

15. The apparatus of claim 14, wherein the program performs instructions to generate the composite feature vector by:
generating a first composite feature vector from the image feature vector and a first text feature vector related to an instrument to generate a first mask for an instrument determined to be present in the task image; and
generating a second composite feature vector from the image feature vector and the second text feature vector relating to a location of the instrument determined to be present in the task image to generate a second mask for the instrument determined to be present in the task image.

16. The apparatus of claim 15, wherein the program performs instructions to generate the first mask by:
generating the first composite feature vector by performing multi-modal fusion of a first text feature vector extracted based on at least one text input related to a name or attribute of an instrument and the image feature vector; and
generating a segmentation mask corresponding to an instrument whose probability of presence in the task image is greater than or equal to a first reference value among instruments input via text by referencing the generated first composite feature vector.

17. The apparatus of claim 15, wherein the program performs instructions to generate the second mask by:
generating a second composite feature vector by performing multi-modal fusion of the second text feature vector extracted based on text input regarding the location of the instrument derived using the first mask and the image feature vector, and
generating a segmentation mask corresponding to an instrument whose probability of presence in the task image is greater than or equal to a second reference value among instruments input via text by referencing the generated second composite feature vector.

18. The apparatus of claim 14, wherein the program performs instructions to detect the instrument present in the task image by:
calculating a single mask by fusing a plurality of masks generated for an instrument determined to be present in the task image using the generated composite feature vector, and
outputting a segmentation map for the instrument present in the task based on the calculated single mask.

19. The apparatus of claim 14, wherein the program further includes instructions to:
acquire time-series feature information by extracting feature vectors from individual task images constituting a task video; and
classify a current task process from the acquired time-series feature information.

20. The apparatus of claim 19, wherein the program further includes instructions to receive the acquired time-series feature information and the classified current task process and predict a next task procedure using at least one of locations, distances, and relationships among instruments detected in a current task image.
